(19) United States
(12) Patent Application Publication
(10) Pub. No.: US 2010/0256243 A1
(43) Pub. Date: Oct. 7, 2010

(54) Dosing Regimen for the Treatment of Alzheimer's Disease With Sulfonyl Fluorides

(76) Inventors: Federica Pericle, El Paso, TX (US); Enrico Braglia, Montagnola (CH)

Correspondence Address: FULBRIGHT & JAWORSKI L.L.P., 600 CONGRESS AVE., SUITE 2400, AUSTIN, TX, 78701, US

(21) Appl. No.: 12/589,523

(22) Filed: Apr. 3, 2009

(51) Int. Cl. A61K 31/10; A61P 25/28

(52) U.S. Cl. 514/711

(57) ABSTRACT

The present invention provides for improved dosing regimens for the provision of sulfonyl fluorides, such a methanesulfonyl fluoride (MSF) to subjects suffering from Alzheimer's Disease (AD). The methods rely on an alternating day dosing scheme that avoids toxicity associated with a daily dosing regimen.

### BACKGROUND OF THE INVENTION

A. Field of the Invention

The present invention relates generally to the fields of neurology and the pharmacotherapy. In particular, the present invention provides dosing regiments for treatment of Alzheimer's Disease with sulfonyl fluorides, including methanesulfonyl fluoride (MSF).

B. Description of the Related Art

Methanesulfonyl fluoride (MSF) is a long-acting irreversible inhibitor of acetylcholinesterase that shows excellent selectivity for the CNS (Moss et al., 1988; Moss et al., 1985). This selectivity seems to be due, in part, to the irreversible mechanism of action. Recovery from irreversible inhibition is a simple function of the rate of new synthesis of acetylcholinesterase in each tissue. Fortunately, acetylcholinesterase in the brain is resynthesized at a rate much slower than peripheral tissues (Moss et al., 1988; Moss et al., 1985). It has been reported that methanesulfonyl fluoride can be used to accumulate up to 80-90% inhibition of rodent and monkey brain acetylcholinesterase with minimum inhibition of peripheral enzyme and without toxicity by using relatively small doses of the drug over a long period of time (Moss et al., 1988; Moss et al., 1985).

In a Phase II study, patients with AD were treated with oral MSF at 0.18 mg/kg every 2 or 3 days (3 times per week). This regimen was dictated by the feasibility of the study and as a convenience for the research project. More specifically, the drug had to be made fresh each day (e.g., absence of stability data) and patients could not be brought to the doctor on a daily basis. The dose of MSF was calculated by producing a minimum level of 50% inhibition of brain AChE when given three times over the course of one week. However, the optimal level of inhibition (90%) would have been achieved by daily administration of MSF based upon the preclinical toxicology data available, and thus this would have been considered the regimen of choice. Moreover, the only dose used in this trial was calculated theoretically and not dictated by preclinical or clinical testing. As per the Author comment in Moss et al. (1999): “Without dose-limiting side effects, our strategy was simply to use a theoretically adequate dose. We made no attempt to find a best dose and the benefit of higher or lower doses were not explored.” A typical Phase II trial in AD patients is conducted after dose-limiting Phase I trial is performed. Moreover, drug doses are calculated based on preclinical studies that in the case of MSF were conducted daily (like all AChE inhibitors) and not 2 or 3 times per week.

### SUMMARY OF THE INVENTION

Thus, in accordance with the present invention, there is provided a method of chronically treating a subject with sulfonyl fluoride (SF) comprising administering a dose of SF to said subject on alternating days for at least 26 weeks. The subject may be a mouse, rat or monkey, or a human. The subject may suffer from Alzheimer's Disease.

The dose of SF may be about 0.07-2 mg/kg. The SF may be administered for a period of six months, nine months, twelve months, eighteen months, two years, three years, four years, five years or ten years. The SF may be methanesulfonyl fluoride. The SF may be administered orally. The SF may be diluted to about 1 mg/ml in a pharmaceutical carrier. The pharmaceutical carrier may be peanut oil.

In another embodiment, there is provided a method of chronically treating a human subject with Alzheimer's Disease (AD) comprising administering to to said subject a dose of sulfonyl fluoride (SF) on alternating days for at least 26 weeks. The subject may have early onset AD or sporadic AD. Treating may comprise improving one or more of short-term memory, long-term memory, abstract thinking, mood, cognitive task performance, speech, and/or orientation.

The dose of SF may be about 0.07-2 mg/kg. The SF may be administered for a period of six months, nine months, twelve months, eighteen months, two years, three years, four years, five years or ten years. The SF may be methanesulfonyl fluoride. The SF may be administered orally. The SF may be diluted to about 1 mg/ml in a pharmaceutical carrier. The pharmaceutical carrier may be peanut oil.

In still yet a further embodiment, there is provided a method of treating a subject having suffered a stroke with sulfonyl fluoride (SF) comprising administering a dose of SF to said subject on alternating days. The subject may be a mouse, rat or monkey, or a human. The dose of SF may be about 0.07-2 mg/kg. The SF may be administered for a period of one week, two weeks, three week, four weeks, one month, two months or three months. The SF may be methanesulfonyl fluoride. The SF may be administered orally. The SF may be diluted to about 1 mg/ml in a pharmaceutical carrier. The pharmaceutical carrier may be peanut oil.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### DETAILED DESCRIPTION OF THE INVENTION

### I. The Present Invention

Methanesulfonyl fluoride (MSF), a highly selective CNS inhibitor of acetylcholinesterase, has been demonstrated to promote improvement in cognitive performance in patients with senile dementia of Alzheimer type. See U.S. Pat. No. 5,798,392. In addition, MSF treatment produced about 90% inhibition of acetylcholinesterase in the brain in cerebrally ischemic animals, and improved cognitive performance as compared to untreated controls. Moreover, whereas brains from both groups of animals revealed similar extent and degree of cerebral infarction, the MSF-treated animals showed more intense septal choline acetyltransferase immunoreactivity than the vehicle-treated animals. These results, reported in U.S. Patent Publication 2003/0087959, showed that MSF, possibly by preserving a functional cholinergic system, attenuated stroke-induced cognitive deficits.

Ex vivo studies of MSF have shown up to 80% inhibition of brain AChE in mice (Kobayashi et al., 1999), rats (Moss et al., 1985), and monkey cortex (Moss et al., 1988) without toxicity. In vitro experiments have shown that MSF is highly selective for inhibition of human cortex AChE compared to butyrylcholinesterase (Pacheco et al., 1995). In vivo, MSF is highly effective in reversing scopolamine-induced amnesia in rats (Palacios-Esquivel et al., 1993), normal age-related memory impairment in aged rats (Malin et al., 1993), and ischemia-induced cognitive deficits in rats (Borlongan et al., 2005). All these studies were conducted using daily doses of MSF given orally or intramuscularly. These data support the use of oral MSF given daily at low doses like all other oral AChE inhibitors on the market (e.g., Aricept®, Exelon®).

An acute toxicology study was conducted with MSF in rats to complete the pre-clinical package for an IND on Alzheimer's Disease (AD). The FDA requires that MSF be tested in two species for 28 days (daily) at 10× the therapeutic dose that will be used in humans. The therapeutic dose was calculated from the clinical testing previously conducted (Moss et al., 1999). In this Phase II study, patients with AD were treated three times a week with oral MSF at 0.18 mg/kg. The regimen was dictated by the feasibility of the study and not scientific reasons, as demonstrated above. The drug had to be made fresh each day (e.g., absence of stability data) and patients could not be brought to the doctor daily. The dose of MSF was calculated by producing a minimum level of 50% inhibition of brain AChE when given three times.

However, preclinical data showed that daily treatment of oral MSF at 0.5 mg/kg produced 90% inhibition in the brain only after 5 days of administration. Based upon the preclinical evidence and the clinical testing, the inventors calculated the therapeutic dose for oral MSF of 0.077 mg/kg/daily (the equivalent of 0.18 mg/kg for three times a week). They then calculated 10× the dose of 0.7 mg/kg and rounded it up to 1 mg/kg. Two experiments were conducted: first, to show the safety of the compound given daily at 10× the therapeutic dose in accordance with the FDA guidelines; and second, to prove that MSF can only be given using the unique regimen of on-and-off instead of daily regimen. As shown below in the Examples, an every-other-day regimen surprisingly produced far less toxicity than the every day schedule at the 10× “rounded-up” dose.

In light of these experimental results, the inventors went back to published data on sulfonyl fluoride compounds in an effort to understand the toxic nature of oral MSF given daily. In a prior publication, the time course of cholinesterase inhibition produced in rats by one injection of phenylmethylsulfonyl fluoride (PMSF) in various tissues was illustrated (FIG. 2, Moss et al., 1988). The heart, ileum and pectoral tissue needed at least 24-48 hours to synthesize novel cholinesterase fundamental for normal function. If the peripheral tissue does not rest at least one day—i.e., enough to recover from the AChE inhibition induced by the drug—the heart, GI tract and muscle would stop functioning, resulting in death. On the contrary, by giving an “on-and-off” administration of the drug, cholinesterase inhibition will occur in the brain where intended, and avoid effects in the periphery (where toxicity arises). One day would be sufficient time for the tissue in the periphery to synthesize new enzyme, thus compensating for the drug blockage. It is also important to stress that vital organs (e.g., heart) have a substantial excess of cholinesterase above the amount required for normal function, and less than 50% inhibition is generally regarded as non-pharmacologically significant (Brimblecombe, 1974). This regimen, which is distinct from the oral, daily regimen for all AChEs (MSF, metrifonate) on the market, is therefore responsible greatly reduced toxicity.

### II. Alzheimer's Disease

AD is a progressive, neurodegenerative disease characterized by memory loss, language deterioration, impaired visuospatial skills, poor judgment, indifferent attitude, but preserved motor function. AD usually begins after age 65, however, its onset may occur as early as age 40, appearing first as memory decline and, over several years, destroying cognition, personality, and ability to function. Confusion and restlessness may also occur. The type, severity, sequence, and progression of mental changes vary widely. The early symptoms of AD, which include forgetfulness and loss of concentration, can be missed easily because they resemble natural signs of aging. Similar symptoms can also result from fatigue, grief, depression, illness, vision or hearing loss, the use of alcohol or certain medications, or simply the burden of too many details to remember at once.

There is no cure for AD and no way to slow the progression of the disease. For some people in the early or middle stages of the disease, medication such as tacrine may alleviate some cognitive symptoms. Aricept (donepezil) and Exelon (rivastigmine) are reversible acetylcholinesterase inhibitors that are indicated for the treatment of mild to moderate dementia of the Alzheimer's type. Also, some medications may help control behavioral symptoms such as sleeplessness, agitation, wandering, anxiety, and depression. These treatments are aimed at making the patient more comfortable.

AD is a progressive disease. The course of the disease varies from person to person. Some people have the disease only for the last 5 years of life, while others may have it for as many as 20 years. The most common cause of death in AD patients is infection.

The molecular aspect of AD is complicated and not yet fully defined. As stated above, AD is characterized by the formation of amyloid plaques and neurofibrillary tangles in the brain, particularly in the hippocampus which is the center for memory processing. Several molecules contribute to these structures: amyloid β protein (Aβ), presenilin (PS), cholesterol, apolipoprotein E (ApoE), and Tau protein. Of these, Aβ appears to play the central role.

Aβ contains approximately 40 amino acid residues. The 42 and 43 residue forms are much more toxic than the 40 residue form. Aβ is generated from an amyloid precursor protein (APP) by sequential proteolysis. One of the enzymes lacks sequence specificity and thus can generate Aβ of varying (39-43) lengths. The toxic forms of Aβ cause abnormal events such as apoptosis, free radical formation, aggregation and inflammation. Presenilin encodes the protease responsible for cleaving APP into Aβ. There are two forms—PS1 and PS2. Mutations in PS1, causing production of Aβ₄₂, are the typical cause of early onset AD.

Cholesterol-reducing agents have been alleged to have AD-preventative capabilities, although no definitive evidence has linked elevated cholesterol to increased risk of AD. However, the discovery that Aβ contains a sphingolipid binding domain lends further credence to this theory. Similarly, ApoE, which is involved in the redistribution of cholesterol, is now believed to contribute to AD development. As discussed above, individuals having the ApoE4 allele, which exhibits the least degree of cholesterol efflux from neurons, are more likely to develop AD.

Tau protein, associated with microtubules in normal brain, forms paired helical filaments (PHFs) in AD-affected brains which are the primary constituent of neurofibrillary tangles. Recent evidence suggests that Aβ proteins may cause hyperphosphorylation of Tau proteins, leading to disassociation from microtubules and aggregation into PHFs.

### III. Stroke

A stroke is defined the rapidly developing loss of brain function(s) due to a disturbance in the blood supply to the brain. This can be due to ischemia (lack of blood supply), caused by thrombosis or embolism, or due to a hemorrhage. As a result, the affected area of the brain is unable to function, leading to inability to move one or more limbs on one side of the body, inability to understand or formulate speech or inability to see one side of the visual field. Strokes are the leading cause of adult disability in the United States and Europe. Risk factors for stroke include advanced age, hypertension (high blood pressure), previous stroke or transient ischemic attack (TIA), diabetes, high cholesterol, cigarette smoking and atrial fibrillation.

The traditional definition of stroke, devised by the World Health Organization in the 1970s, is a “neurological deficit of cerebrovascular cause that persists beyond 24 hours or is interrupted by death within 24 hours.” This definition was supposed to reflect the reversibility of tissue damage and was devised for the purpose, with the time frame of 24 hours being chosen arbitrarily. The 24-hour limit divides stroke from transient ischemic attack, which is a related syndrome of stroke symptoms that resolve completely within 24 hours. With the availability of treatments that, when given early, can reduce stroke severity, many now prefer alternative concepts, such as brain attack and acute ischemic cerebrovascular syndrome (modeled after heart attack and acute coronary syndrome respectively), that reflect the urgency of stroke symptoms and the need to act swiftly.

Strokes can be classified into two major categories: ischemic and hemorrhagic. Ischemia is due to interruption of the blood supply, while hemorrhage is due to rupture of a blood vessel or an abnormal vascular structure. 80% of strokes are due to ischemia; the remainder are due to hemorrhage. Some hemorrhages develop inside areas of ischemia (“hemorrhagic transformation”).

In an ischemic stroke, blood supply to part of the brain is decreased, leading to dysfunction of the brain tissue in that area. There are four reasons why this might happen: thrombosis, embolism, systemic hypoperfusion and venous thrombosis. Stroke without an obvious explanation is termed “cryptogenic”; this constitutes 30-40% of all ischemic strokes.

Intracranial hemorrhage is the accumulation of blood anywhere within the skull vault. A distinction is made between intra-axial hemorrhage (blood inside the brain) and extra-axial hemorrhage (blood inside the skull but outside the brain). Intra-axial hemorrhage is due to intraparenchymal hemorrhage or intraventricular hemorrhage (blood in the ventricular system). The main types of extra-axial hemorrhage are epidural hematoma (bleeding between the dura mater and the skull), subdural hematoma (in the subdural space) and subarachnoid hemorrhage (between the arachnoid mater and pia mater). Most of the hemorrhagic stroke syndromes have specific symptoms (e.g. headache, previous head injury). Intracerebral hemorrhage (ICH) is bleeding directly into the brain tissue, forming a gradually enlarging hematoma (pooling of blood). In thrombotic stroke, a thrombus (blood clot) usually forms around atherosclerotic plaques. Since blockage of the artery is gradual, onset of symptomatic thrombotic strokes is slower. A thrombus itself (even if non-occluding) can lead to an embolic stroke (see below) if the thrombus breaks off, at which point it is called an embolus.

An embolic stroke refers to the blockage of an artery by an embolus, a travelling particle or debris in the arterial bloodstream originating from elsewhere. An embolus is most frequently a thrombus, but it can also be a number of other substances including fat (e.g., from bone marrow in a broken bone), air, cancer cells or clumps of bacteria (usually from infectious endocarditis). Thus, the source of the embolus must be identified. Because the embolic blockage is sudden in onset, symptoms usually are maximal at start. Also, symptoms may be transient as the embolus is partially resorbed and moves to a different location or dissipates altogether. Emboli most commonly arise from the heart (especially in atrial fibrillation) but may originate from elsewhere in the arterial tree. In paradoxical embolism, a deep vein thrombosis embolises through an atrial or ventricular septal defect in the heart into the brain.

Systemic hypoperfusion is the reduction of blood flow to all parts of the body. It is most commonly due to cardiac pump failure from cardiac arrest or arrhythmias, or from reduced cardiac output as a result of myocardial infarction, pulmonary embolism, pericardial effusion, or bleeding. Hypoxemia (low blood oxygen content) may precipitate the hypoperfusion. Because the reduction in blood flow is global, all parts of the brain may be affected, especially “watershed” areas—border zone regions supplied by the major cerebral arteries. Blood flow to these areas does not necessarily stop, but instead it may lessen to the point where brain damage can occur. This phenomenon is also referred to as “last meadow” to point to the fact that in irrigation the last meadow receives the least amount of water.

Cerebral venous sinus thrombosis leads to stroke due to locally increased venous pressure, which exceeds the pressure generated by the arteries. Infarcts are more likely to undergo hemorrhagic transformation (leaking of blood into the damaged area) than other types of ischemic stroke.

Intracerebral hemorrhage generally occurs in small arteries or arterioles and is commonly due to hypertension, trauma, bleeding disorders, amyloid angiopathy, illicit drug use (e.g., amphetamines or cocaine), and vascular malformations. The hematoma enlarges until pressure from surrounding tissue limits its growth, or until it decompresses by emptying into the ventricular system, CSF or the pial surface. A third of intracerebral bleed is into the brain's ventricles. ICH has a mortality rate of 44 percent after 30 days, higher than ischemic stroke or even the very deadly subarachnoid hemorrhage.

### IV. Sulfonyl Fluorides

Methanesulfonyl fluoride (MSF) is in the class of drugs known as sulfonyl fluorides, which also includes ethanesuflonyl fluoride. It is a long-lasting CNS selective cholinesterase inhibitor developed for the treatment of senile dementia of the Alzheimer type (SDAT) (Moss et al., 1988). It reacts with cholinesterase as an irreversible inhibitor that forms a covalent sulfonyl-enzyme complex. This complex inhibits the activity of cholinesterase and seems more effective in the brain than in the periphery (e.g., heart, GI, muscle). It has been showed that MSF produced an average of 90% inhibition of brain cholinesterase with less than 30% inhibition of enzyme in the periphery when the drug was administered daily at low doses in rodents (Moss et al., 1988). MSF action is determined by de novo synthesis of AChE. MSF exploits the extremely slow de novo synthesis in brain (t_{1/2} of 12 days), compared to peripheral tissues (e.g., smooth muscle t_{1/2} of 1 day), to achieve exceptionally selective inhibition of brain AChE without peripheral toxicity (e.g., nausea, vomiting, and diarrhea). Although MSF has high potential toxicity (common to all powerful AChE inhibitors), when diluted and dosed therapeutically, its high potency and selectivity produce extraordinary efficacy.

Another drug that may be substituted for SFs in the methods of the present invention is metrifonate (trichlorfon), an irreversible organophosphate acetylcholinesterase inhibitor. It has been used to treat schistosomiasis caused by Schistoma haematobium but is no longer commercially available. It has been proposed for use in treatment of Alzheimer's disease, though not currently approved for that indication.

### V. Pharmaceutical Compositions and Routes of Administration

Pharmaceutical compositions containing a sulfonyl fluoride may contain one or more pharmaceutical carriers. The term “pharmaceutically acceptable carrier” refers to any generally acceptable excipient that is relatively inert, non-toxic and non-irritating. Because sulfonyl fluorides are minimally soluble and relatively unstable in water, the product should be dissolved in an oil miscible carrier. A sulfonyl fluoride also may be administered in an emulsion. When the carrier serves as a diluent, it may be solid, semisolid, or liquid material acting as a vehicle, excipient, or medium for the active ingredient. Pharmaceutical unit dosage forms may be prepared for administration by any of several routes, including, but not limited to, oral and parenteral (especially by intramuscular and intravenous injection (in a vehicle other than oil), or by subcutaneous implant or transdermal administration.). Representative of such forms are tablets, soft and hard gelatin capsules, powders, lozenges, chewing gums, emulsions, suspensions, syrups, solutions, sterile injectable solutions, and sterile packaged powders. Composition containing a sulfonyl fluoride may be formulated by procedures known in the art so as to provide rapid, sustained, or delayed release of any or all of the compounds after administration.

As the sulfonyl fluoride formulation of the present invention is well suited to oral administration, preferred carriers will facilitate formulation in tablet or capsule form. Solid pharmaceutical excipients such as magnesium stearate, calcium carbonate, silica, starch, sucrose, dextrose, polyethylene glycol (PEG), talc, and the like may be used with other conventional pharmaceutical adjuvants including fillers, lubricants, wetting agents, preserving agents, disintegrating agents, flavoring agents, and binders such as gelatin, gum arabic, cellulose, methylcellulose, and the like to form admixtures which may be used as such or may be tabulated, encapsulated, or prepared in other suitable forms as noted above. A general description of formulation is given in Remington's Pharmaceutical Sciences.

Administration is preferably by oral dosage but may be by transdermal application, intranasal spray, bronchial inhalation, parenteral injection (e.g., intramuscular or intravenous injection), and the like. Carriers for parenteral administration include, without limitation, aqueous solutions of dextrose, mannitol, mannose, sorbitol, saline, pure water, ethanol, glycerol, propylene glycol, peanut oil, sesame oil, polyoxyethylene-polyoxypropylene block polymers, and the like. One may additionally include suitable preservatives, stabilizers, antioxidants, antimicrobials and buffering agents, for example BHA, BHT, citric acid, ascorbic acid, tetracycline, and the like.

### VI. Combination Therapies

In order to increase the effectiveness of the sulfonyl fluoride therapy of the present invention, it may be desirable to combine these compositions with other agents effective in the treatment of AD and stroke. Compositions would be provided in a combined amount effective to confer a therapeutic benefit to a person suffering from AD and stroke. This process may involve administering the sulfonyl fluoride and the second agent(s) to the subject at the same time, for example, using a single composition or pharmacological formulation that includes both agents, or using two distinct compositions or formulations given at the same time, wherein one composition includes the sulfonyl fluoride and the other includes the second agent(s). Alternatively, the second agent therapy may precede or follow the sulfonyl fluoride treatment by intervals ranging from minutes to weeks.

The exact schedule of treatment with sulfonyl fluorides and second agent therapy is determined in large part by the pharmacokinetic or pharmacodynamic properties of the sulfonyl fluoride and the second agents. The sulfonyl fluorides typically have long pharmacodynamic effects with half-life times in hours or days, whereas the second agents have much shorter pharmacokinetic and pharmacodynamic effects, in the range of minutes to hours. These differences would dictate the most efficacious administration schedules and routes of administration.

In embodiments where the other agent and sulfonyl fluoride are administered separately to the subject, one may wish that a significant period of time did not expire between the time of each delivery, such that the second agent and sulfonyl fluoride would be able to exert an advantageously combined effect on the subject. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, sulfonyl therapy is “A” and the second agent is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

Administration of the second agent will follow general protocols for the administration of sulfonyl fluoride, taking into account the toxicity, if any, of the agent. It is expected that the treatment cycles would be repeated as necessary. In one embodiment, it is envisioned that both drugs are given in the same alternating daily regiment, while another embodiment would comprise delivering the second agent on the “off day” when MSF is not administered.

Drugs that can be used in conjunction with MSF in accordance with the AD aspects of the present invention include cholinesterase inhibitors that prevent the breakdown of acetylcholine. Three commonly prescribed cholinesterase inhibitors include donepezil (Aricept®), approved to treat all stages of AD, rivastigmine (Exelon®), approved to treat mild to moderate AD, and galantamine (Razadyne®), approved to treat mild to moderate AD. Memantine (Namenda®) works by regulating the activity of glutamate, a different messenger chemical involved in learning and memory. It was approved in 2003 for treatment of moderate to severe AD.

Medical therapies aimed at minimizing clot enlargement or preventing new clots from forming may be used in conjunction with MSF to treat stroke in accordance with the present invention. Medications such as aspirin, clopidogrel and dipyridamole may be given to prevent platelets from aggregating. Pharmacologic thrombolysis (“clot busting”) with tissue plasminogen activator (tPA) and therapeutic hypothermia are other adjunct therapies.

### VII. Kits

All the essential materials and reagents required for treatment of patients with a sulfonyl fluoride may be assembled together in a kit. This generally will comprise a the drug in a form suitable to stable storage, optionally include any excipients required for administration. Instructions for administration may also be included.

The container means of the kits will generally include at least one vial, test tube, flask, bottle, or other container means, into which a component may be placed, and preferably, suitably aliquoted. Where there is more than one component in the kit, the kit also will generally contain additional containers into which the additional components may be separately placed. However, various combinations of components may be comprised in a container. The kits of the present invention also will typically include a means for packaging the component containers in close confinement for commercial sale. Such packaging may include injection or blow-molded plastic containers into which the desired component containers are retained.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Daily Dosing Regimen

Daily dosing with 1 mg/kg MSF in rats resulted in high toxicity and animal deaths, as shown below in Table 1. These results suggested that 1 mg/kg was too toxic or MSF may not be administered daily in contrast with other AChE inhibitors.

TABLE 1
Initial Protocol

| | |
|---|---|
| Title: | Daily dosing with 1.0 mg/kg MSF/peanut oil in rats |
| Background: | A starting point was needed to ascertain a viable dose of MSF/peanut |
| | oil in rats. |
| Objective: | Conduct a non-GLP study to determine a dose response using gross |
| | clinical observations. |
| Design: | Administration: oral gavage, 1.0 mg/kg daily |
| | Primary outcome parameter: clinical signs of distress and/or death. |
| | Number of animals: 7 animals (4 males, 3 females). Rats used were |
| | naïve rats left over from other Stillmeadow studies. Rats will be |
| | weighed at onset of study and dosed according to regimen above, with |
| | a MSF/peanut oil concentration of 1.0 mg/ml, at a dose rate of 1.0 ml/kg. |
| Study center: | Stillmeadow, Inc. |
| Results: | By Day 2, some rats showed signs of decreased activity. One rat was |
| | Found Dead (FD) on Day 3. The remainder of the rats were FD on |
| | Day 4. |

TABLE 2
MSF given orally and daily at sub-toxic doses is lethal

| Dose (mg/kg)/daily | Duration* | Number Alive/Numbered Treated |
|---|---|---|
| 1 | 4 days | 0/7 (4 males + 3 female) |

*The experiment was designed for 28-day study, however after 4 days all animals were found dead

### Example 2

The same total amount of MSF given in Example 1 (2 mg/kg twice for a total of 4 mg/kg) was given every other day, and surprisingly resulted in no lethal event (Table 3). These data indicated that MSF should only be administered in a “pulsing” and not daily regimen.

To confirm the “pulsing” theory for MSF administration, the inventor continued the experiment described above and extended MSF treatment to double the total lethal dose observed in Example 1 (from 4 mg/kg to 8 mg/kg). Rats in were treated at 2 mg/kg/every other day for 2 more round (total of 8 mg/kg in 8 days). 83.3% of the animals survived (⅚), and were still alive and well after more than two weeks of follow-up observation.

TABLE 3
MSF given orally every other day is not lethal

| | | Number Alive/Numbered |
|---|---|---|
| Dose (mg/kg)/every other day | Duration | Treated |
| 2 | 4 days | 6/6 (3 males + 3 female) |

TABLE 4
Double the lethal dose of MSF given orally every other
day is sub-lethal

| | | Number Alive/Numbered |
|---|---|---|
| Dose (mg/kg)/every other day | Duration | Treated |
| 2 | 8 days | 5/6 (3 males + 3 female) |

TABLE 5
MSF given orally every other day is not lethal

| | |
|---|---|
| Title: | Alternating day dosing with 2.0 mg/kg MSF/peanut oil in rats for 4 |
| | days (total of 2 doses = 4 mg/kg) |
| Background: | MSF/peanut oil at 1 mg/kg/day is lethal after 4 days of treatment. |
| Objective: | A modified dosing regimen was needed to avoid toxicity observed in the |
| | initial protocol. |
| Design: | Administration: oral gavage, 2.0 mg/kg every other day for 4 days (total of |
| | 2 doses = 4 mg/kg) |
| | Primary outcome parameter: death. |
| | Number of animals: 6 (3 female + 3 male) naïve rats were used in this |
| | study. Rats were weighed at onset of study and dosed according to regimen |
| | above, with a MSF/peanut oil concentration of 2 mg/kg. |
| Study center: | Stillmeadow, Inc. |
| Results: | By Day 4, all rats were alive and it was decided to continue the treatment |
| | for 4 more days with 2 more treatments of 2 mg/kg each for a total of 8 mg/kg |
| | (twice the total lethal dose of 4 mg/kg in 4 days at 1 mg/kg/day). |
| | After 7 days 1 animal was found dead but 5/6 completed the regimen and |
| | were alive and well for over two weeks of follow-up observation |

TABLE 6
Acute oral toxicity (LD₅₀) of MSF in animals

| | | Number Dead/Number | | |
|---|---|---|---|---|
| Dose Level | | Treated | | |
| (mg/kg) | (mg/mL) | Males | Females | Combination |
| 1 | 0.4 | 0/5 | 0/5 | 0/10 |
| 2 | 0.8 | 0/5 | 0/5 | 0/10 |
| 3 | 1.2 | 0/5 | 1/5 | 1/10 |
| 4 | 1.6 | 5/5 | 5/5 | 10/10 |

The acute oral LD₅₀, as indicated by the data, was determined to be greater than 3 mg/kg but less than 4 mg/kg.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### IX. References

The following references, to the extent that they provide exemplary procedural or other details supplementary to those set forth herein, are specifically incorporated herein by reference.

U.S. Pat. No. 5,798,392

### U.S. Patent Publn. 2003/0087959

Borlongan et al., Brain Res., 1038(1):50-58, 2005.
Brimblecombe, In: Drug Actions on Cholinergic Systems, Macmillan (London), 227, 1974.
Kobayashi et al., J Health Sci., 45(4):191-202, 1999.
Malin et al. Neurobiology of Aging, 14:393-395, 1993.
Moss et al., Alzheimer Dis. Assoc. Disord., 13(1):20-25, 1999.
Moss et al., In: Current Research in Alzheimer therapy: Cholinesterase Inhibitors, Giacobini and Becker (Eds.), Taylor and Francis, NY, 305-314, 1988.
Moss et al., In: Neurology and Neurobiology 18: Senile Dementia of the Alzheimer Type, Hutton and Kenny (Eds.), Allan R. Liss, NY, 337-350, 1985.
Pacheco et al., J. Pharmacol. Experim. Therapeutics, 274:767-770, 1995.
Palacios-Esquivel et al., Neurobiology of Aging, 14:93-96, 1993.

What is claimed is:

1. A method of chronically treating a subject with sulfonyl fluoride (SF) comprising administering a dose of SF to said subject on alternating days for at least 26 weeks.

2. The method of claim 1, wherein said subject is a mouse, rat or monkey.

3. The method of claim 1, wherein said subject is a human.

4. The method of claim 3, wherein said subject suffers from Alzheimer's Disease.

5. The method of claim 1, wherein said dose of SF is about 0.07-2 mg/kg.

6. The method of claim 1, wherein SF is administered for a period of six months, nine months, twelve months, eighteen months, two years, three years, four years, five years or ten years.

7. The method of claim 1, wherein SF is methanesulfonyl fluoride.

8. The method of claim 1, wherein SF is administered orally.

9. The method of claim 8, wherein SF is diluted to about 1 mg/ml in a pharmaceutical carrier.

10. The method of claim 9, wherein said pharmaceutical carrier is peanut oil.

11. A method of chronically treating a human subject with Alzheimer's Disease (AD) comprising administering to to said subject a dose of sulfonyl fluoride (SF) on alternating days for at least 26 weeks.

12. The method of claim 11, wherein said subject has early onset AD.

13. The method of claim 11, wherein said subject has sporadic AD.

14. The method of claim 11, wherein treating comprises improving one or more of short-term memory, long-term memory, abstract thinking, mood, cognitive task performance, speech, and/or orientation.

15. The method of claim 11, wherein said dose of SF is about 0.07-2 mg/kg.

16. The method of claim 11, wherein SF is administered for a period of six months, nine months, twelve months, eighteen months, two years, three years, four years, five years or ten years.

17. The method of claim 11, wherein SF is methanesulfonyl fluoride.

18. The method of claim 11, wherein SF is administered orally.

19. The method of claim 18, wherein SF is diluted to about 1 mg/ml in a pharmaceutical carrier.

20. The method of claim 19, wherein said pharmaceutical carrier is peanut oil.
